(19) United States
(12) Patent Application Publication
(10) Pub. No.: US 2024/0325975 A1
(43) Pub. Date: Oct. 3, 2024

(54) FILTER MODULE AND AIR PURIFIER WITH THE SAME

(71) Applicants: Jeoungmin LEE, Seoul (KR); SWIS CO., LTD, Chenan-si, Chungcheongnam-do (KR)

(72) Inventor: Jeoungmin LEE, Seoul (KR)

(73) Assignees: Jeoungmin LEE, Seoul (KR); SWIS CO., LTD, Chenan-si, Chungcheongnam-do (KR)

(21) Appl. No.: 18/580,419

(22) PCT Filed: Jul. 27, 2021
(86) PCT No.: PCT/KR2021/009744

§ 371 Date: Jan. 18, 2024

(30) Foreign Application Priority Data
Jul. 21, 2021 (KR) 10-2021-0095983

(51) Int. Cl. B01D 53/88; A61L 9/12; A61L 9/20; B01D 46/00; B01D 46/24; B01D 53/00; B01J 21/06; B01J 23/755; B01J 35/56

(52) U.S. Cl. CPC B01D 53/885; A61L 9/12; A61L 9/205; B01D 46/0028; B01D 46/0038; B01D 46/0047; B01D 46/2403; B01D 53/007; B01J 21/063; B01J 23/755; B01J 35/56; A61L 2209/12; A61L 2209/133; A61L 2209/14; B01D 2255/20707; B01D 2255/802; B01D 2257/90; B01D 2257/91; B01D 2258/06; B01D 2259/804; B01D 2279/65

(57) ABSTRACT

A filter module includes: a dust collection filter, which is a tubular filter having a hollow formed therein, the dust collection filter being configured to filter out contaminants from air flowing from outside an outer circumference thereof toward an interior of the hollow; a photocatalytic filter configured to allow air introduced into the hollow and discharged out of the dust collection filter through an opening above the hollow to pass therethrough; a first filter holder configured to fixedly support the photocatalytic filter and an upper end of the dust collection filter so as not to obstruct air flowing from the interior of the hollow to the photocatalytic filter; and a second filter holder configured to fixedly support a lower end of the dust collection filter.

### CROSS-REFERENCE TO PRIOR APPLICATIONS

This Application is a National Stage Patent Application of PCT International Application No. PCT/KR2021/009744 (filed on Jul. 27, 2021), which claims priority to Korean Patent Application No. 10-2021-0095983 (filed on Jul. 21, 2021), which are all hereby incorporated by reference in their entirety.

### BACKGROUND

The present invention relates to a filter module provided in an air purifier, and more particularly to a filter module including titanium oxide and an air purifier including the same.

An air purifier, which filters out harmful matter from indoor air to clean indoor air, is becoming increasingly necessary as air pollution intensifies. In common, conventional air purifiers include a housing and a filter removably installed in the housing. Among the conventional air purifiers, small air purifiers that are portable or easily relocated have an insufficient air purification function due to the simple structure of the filter. Meanwhile, an air purifier configured to allow air to pass through a plurality of filters in stages must be placed on the floor or fixed to the wall due to the large size and complex structure of the air purifier, and it is difficult to relocate the air purifier. In addition, the manufacturing cost increases.

### SUMMARY

The present invention provides a filter module having a function of removing foreign matter, such as fine dust, yellow dust, and dust, from the air, and a virus removal, odor removal, and sterilization function and an air purifier including the same.

The present invention provides a filter module including a dust collection filter, which is a tubular filter having a hollow formed therein, the dust collection filter being configured to filter out contaminants from air flowing from outside an outer circumference thereof toward the interior of the hollow, a photocatalytic filter configured to allow air introduced into the hollow and discharged out of the dust collection filter through an opening above the hollow to pass therethrough, the photocatalytic filter comprising a pore plate having a plurality of pores formed therein and a titanium oxide layer formed by laminating titanium oxide on a surface of the pore plate through which the air is discharged, a first filter holder configured to fixedly support the photocatalytic filter and an upper end of the dust collection filter so as not to obstruct air flowing from the interior of the hollow to the photocatalytic filter, and a second filter holder configured to fixedly support a lower end of the dust collection filter.

The pore plate may be made of porous nickel foam.

The filter module may further include a hollow closure cap removably coupled to the second filter holder, the hollow closure cap being configured to close an opening under the hollow, wherein the hollow closure cap may include a solid air freshener pocket configured to allow a solid air freshener to be received therein.

The hollow closure cap may further include a downwardly projecting handle, and the filter module may be pulled down along with the handle when the handle is pulled down in the state in which the hollow closure cap is fixedly coupled to the second filter holder.

In addition, the present invention provides an air purifier including a case having an air inlet formed at the side and an air outlet formed at the top, the filter module configured as described above, the filter module being disposed in the case, the filter module being configured to purify air introduced into the case through the air inlet, a fan disposed in the case, the fan being configured to move air such that the air is introduced into the case through the air inlet, passes through the filter module, rises, and is discharged out of the case through the air outlet, and an ultraviolet radiator disposed above the photocatalytic filter so as to face the photocatalytic filter in the case, the ultraviolet radiator including an ultraviolet A light emitting diode (LED) configured to radiate ultraviolet A to the titanium oxide layer such that OH radicals are generated at the titanium oxide layer and an LED printed circuit board (PCB) on which the ultraviolet A LED is electrically mounted.

The LED PCB may be provided with a through-hole configured to allow air to flow therethrough.

The ultraviolet radiator may further include an ultraviolet C LED configured to radiate ultraviolet C used to sterilize air that has passed through the photocatalytic filter, the ultraviolet C LED being electrically mounted on the LED PCB.

The filter module may further include a permanent magnet fixedly installed at the first filter holder, the air purifier may include a permanent magnet disposed above the LED PCB in the case, attractive force being applied between the permanent magnet of the filter module and the permanent magnet disposed above the LED PCB, and the first filter holder may be removably attached to the LED PCB by the attractive force between the permanent magnet of the filter module and the permanent magnet disposed above the LED PCB.

A filter module according to the present invention includes a dust collection filter and a photocatalytic filter configured to allow air that has passed through the dust collection filter to pass therethrough, wherein the photocatalytic filter includes a titanium oxide layer at which OH radicals are generated when ultraviolet A is incident thereon. Consequently, the filter module and the air purifier including the same have excellent performance in removing foreign matter, such as fine dust, yellow dust, and dust, from the air and excellent performance in removing viruses, odor molecules, and harmful bacteria.

### BRIEF DESCRIPTION OF THE DRAWINGS

FIG. 1 is a sectional view of an air purifier according to an embodiment of the present invention.

FIGS. 2 and 3 are exploded perspective views of a filter module and an ultraviolet radiator installed in FIG. 1, wherein

FIG. 2 is a view when viewed from above and FIG. 3 is a view when viewed from below.

FIG. 4 is an enlarged photograph of a pore plate of a photocatalytic filter of FIGS. 2 and 3.

FIG. 5 is a perspective view of a primary dust collection filter of FIG. 1.

### DETAILED DESCRIPTION

The present invention has excellent performance in removing foreign matter, such as fine dust, yellow dust, and dust, from the air and excellent performance in removing viruses, odor molecules, and harmful bacteria.

The present invention may be used in an air purifier and a filter installed in the air purifier to purify air.

Hereinafter, a filter module according to an embodiment of the present invention and an air purifier including the same will be described in detail with reference to the accompanying drawings. The terminology used herein is intended to adequately describe preferred embodiments of the present invention, and may vary depending on the intent of a user or an operator or the conventions of the art to which the present invention pertains. Accordingly, definitions of the terminology should be based on the context of this specification as a whole.

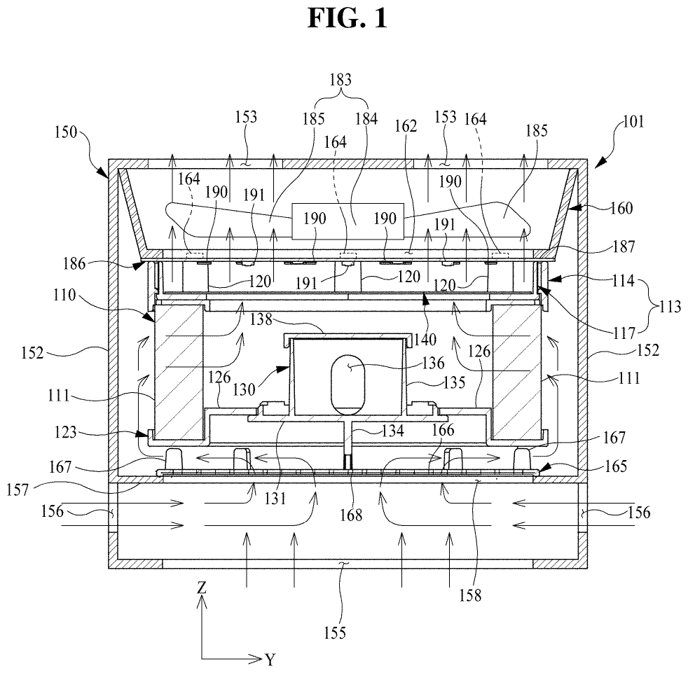

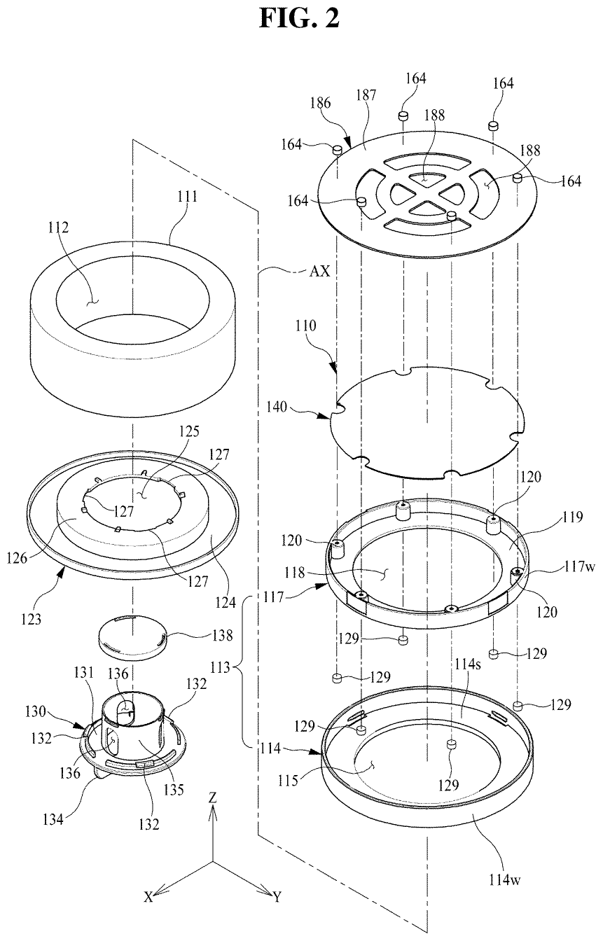

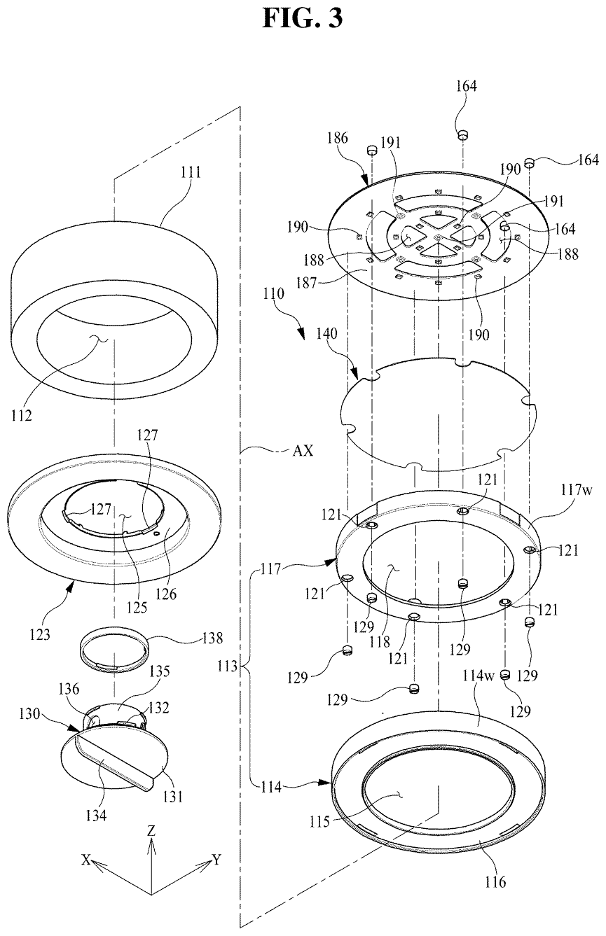

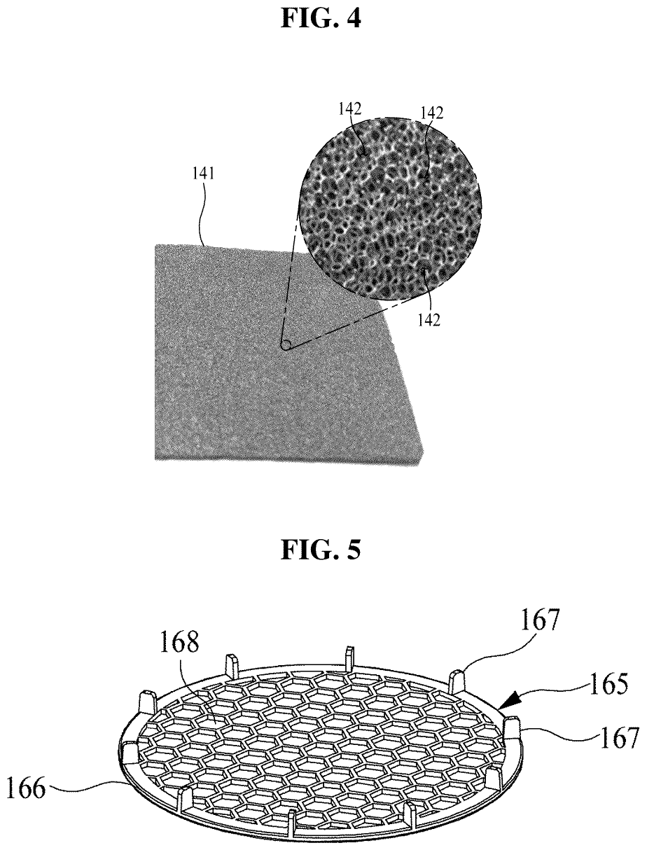

FIG. 1 is a sectional view of an air purifier according to an embodiment of the present invention, FIGS. 2 and 3 are exploded perspective views of a filter module and an ultraviolet radiator installed in FIG. 1, wherein FIG. 2 is a view when viewed from above and FIG. 3 is a view when viewed from below, FIG. 4 is an enlarged photograph of a pore plate of a photocatalytic filter of FIGS. 2 and 3, and FIG. 5 is a perspective view of a primary dust collection filter of FIG. 1. Referring to FIGS. 1 to 5, the air purifier 101 according to the embodiment of the present invention includes a case 150, a filter module 110, a fan 183, and an ultraviolet radiator 186. The air purifier 101 may be used, for example, on the floor or on a flat desk as a so-called stand type, or may be suspended from the ceiling as a ceiling type. Alternatively, the air purifier 101 may be manufactured in small size so as to be used as a portable air purifier. When used as a stand type, purified air is discharged upward from the air purifier 101, and when used as a ceiling type, purified air is discharged downward from the air purifier 101.

The case 150 may be approximately cylindrical or hexagonal in shape, but the case provided in the present invention is not limited to such shapes. The case 150 has an upper end portion provided with an upwardly open air outlet 153 and a lower end portion provided with air inlets 155 and 156. The air inlets 155 and 156 are divided into a bottom wall air inlet 155 provided in a bottom wall of the case 150 and a side wall air inlet 156 provided in a side wall 152 of the case 150.

The case 150 is provided therein with an inner wall 157 configured to separate an inner space of the lower end portion, in which the air inlets 155 and 156 are formed, from a space in which the filter module 110 is located. The fan 183 and the ultraviolet radiator 186 are fixedly installed in the case 150.

In other words, the air purifier 101 includes an ultraviolet radiator support 160 to which the ultraviolet radiator 186 is fixedly attached in the case 150. The ultraviolet radiator support 160 is installed in the case 150.

The case 150 may have a structure in which an upper housing and a lower housing are detachably coupled to each other, but the case provided in the present invention is not necessarily limited to the structure in which the upper housing and the lower housing are separated from each other. The filter module 110 may be inserted into the upper housing from the outside of the upper housing when the upper housing and the lower housing are separated from each other, or conversely, may be removed from the inside of the upper housing to the outside of the upper housing when the upper housing and the lower housing are separated from each other.

An air inlet grill having a plurality of spaced apart grill blades may be installed inside the air inlets 155 and 156 such that the straightness and flow rate of air introduced through the air inlets 155 and 156 are improved. In addition, an air outlet grill having a plurality of spaced apart grill blades may be installed inside the air outlet 153 such that the straightness and flow rate of air discharged from the air outlet 153 to the outside are improved.

The fan 183 is configured to move air such that the air is introduced into the case 150 through the air inlets 155 and 156, passes through the filter module 110, rises, and is discharged out of the case 150 through the air outlet 153, and is disposed above the filter module 110 and the ultraviolet radiator 186 in the case 150. The fan 183 includes a fan propeller 185 configured to discharge air upward from below in a pressurized state and an electric motor 184 configured to provide power necessary to rotate the fan propeller 185.

The filter module 110 is disposed in the case 150 to purify air introduced into the case 150 through the air inlets 155 and 156, and includes a dust collection filter 111, a photocatalytic filter 140, a first filter holder 113, a second filter holder 123, and a hollow closure cap 130. The dust collection filter 111, which is a tubular filter having a hollow 112 formed therein, filters out contaminants from air flowing from outside an outer circumference thereof toward the interior of the hollow 112 to purify air. The dust collection filter 111 may be, for example, a replaceable HEPA filter configured to collect foreign matter using static electricity or a nanofiber filter including a plurality of nanofiber layers configured to filter out foreign matter, the nanofiber filter being configured to be washed out with water, thus allowing for semi-permanent use.

The photocatalytic filter 140 is a filter configured to allow air introduced into the hollow 112 of the dust collection filter 111, rising, and discharged out of the dust collection filter 111 through an opening above the hollow 112 to pass therethrough, and includes a pore plate 141 and a titanium oxide layer formed on a surface of the pore plate by lamination. The pore plate 141 is a plate-shaped member having a plurality of pores 142 formed therein. Air discharged upward from the secondary dust collection filter 111 is introduced into a lower surface of the pore plate 141 and is discharged from an upper surface of the pore plate 141.

The titanium oxide layer is formed by laminating titanium oxide on the surface of the pore plate 141. Methods of laminating titanium oxide on the pore plate 141 include, for example, a sol-gel process, a chemical vapor deposition process, and a sputtering process. The pore plate 141 may be made of a porous foam resin or a blown metal. Preferably, the pore plate 141 is made of porous nickel foam. The porous nickel foam containing nickel (Ni) promotes the improvement of the lamination quality of the titanium oxide, in particular the bonding strength, durability, etc. In addition, the nickel foam promotes the generation of OH radicals when ultraviolet A is incident on the titanium oxide layer laminated thereon. Furthermore, the pore plate 141 formed using the nickel foam has relatively large pore sizes, such as 100 to 300 μm, such that air can easily penetrate the photocatalytic filter 140 in a thickness direction thereof.

The first filter holder 113 is configured to fixedly support the photocatalytic filter 140 and an upper end portion of the dust collection filter so as not to obstruct air flowing from the interior of the hollow 112 so as to penetrate the photocatalytic filter 140, and includes a first member 114 and a second member 117 fixedly coupled to the second member 117 in a state of overlapping the first member 114. Each of the first member 114 and the second member 117 is made of a plastic material.

The second member 117 includes a ring-shaped photocatalytic filter support plate portion 119 having an upper surface configured to support an outer circumferential portion of the photocatalytic filter 140, an annular sidewall 117w bent upward and extending from an outer circumferential edge of the photocatalytic filter support plate portion 119, and six magnet pockets 120 disposed so as to be spaced apart from each other by the same angle based on an axial line AX of the filter module 110. The six magnet pockets 120 protrude upward by the height of the annular sidewall 117w, and each of the six magnet pockets is provided therein with one permanent magnet 129. The six permanent magnets 129 are inserted into and installed in the six magnet pockets 120 through inlets 121 of the six magnet pockets 120 open to a lower surface of the photocatalytic filter support plate portion 119 before the second member 117 is mounted on the first member 114. The photocatalytic filter support plate portion 119 is provided in a central part thereof with a through-hole 118 configured to allow air to flow therethrough.

The first member 114 includes a ring-shaped second member support plate portion 114s having an upper surface configured to support the photocatalytic filter support plate portion 119 in contact therewith and an annular sidewall 114w bent upward and extending from an outer circumferential edge of the second member support plate portion 114s. The second member support plate portion 114s is provided in a lower surface thereof with an upper end fixing recess 116 into which an upper end of the dust collection filter 111 is fitted so as to be fixed thereto. The second member support plate portion 114s is provided in a central part thereof with a through-hole 115 configured to allow air to flow therethrough.

When the first member 114 and the second member 117 are coupled to each other, the height of an upper end of the annular sidewall 114w of the first member 114 is equal to the height of an upper end of the annular sidewall 117w of the second member 117. The annular sidewalls 114w and 117w may function as spacers configured to separate the photocatalytic filter 140 and the ultraviolet radiator 186 from each other.

The second filter holder 123 is configured to fixedly support a lower end portion of the dust collection filter 111, and is an approximately ring-shaped member made of a plastic material. A through-hole, to which the hollow closure cap 130 is coupled, is formed in the center of the second filter holder 123, and a lower end portion fixing recess 124, into which a lower end portion of the dust collection filter 111 is fitted so as to be fixed thereto, is formed in an upper surface of an outer circumferential portion of the second filter holder 123.

The hollow closure cap 130 is removably coupled to the second filter holder 123 so as to close an opening under the hollow 112. When the hollow closure cap 130 is coupled to the second filter holder 123, air introduced into the hollow 112 flows only to the upper side where the photocatalytic filter 140 is disposed. The hollow closure cap 130 includes a disc portion 131 sized to close a through-hole 125 of the second filter holder 123, a solid air freshener pocket 135 formed so as to project upward from the disc portion 131, and a handle 134 formed so as to project downward from the disc portion 131.

The disc portion 131 has a plurality of holder interference protrusions 132 radially bent and extending from an outer circumferential portion thereof. An inner circumferential edge of the second filter holder 123 defining the through-hole 125 is provided with a ring-shaped cap interference inner circumferential portion 126 configured to interfere with the plurality of holder interference protrusions 132. A plurality of radially indented step recesses 127, through which the plurality of holder interference protrusions 132 pass in an upward and downward direction, is formed in the cap interference inner circumferential portion 126.

When a user of the air purifier 101 raises the hollow closure cap 130 from below to above the second filter holder 123 along the axial line AX of the filter module 110 in a state of aligning the plurality of holder interference protrusions 132 and the plurality of step recesses 127 with each other up and down while holding the handle 134, the plurality of holder interference protrusions 132 passes through the plurality of step recesses 127, but the disc portion 131 is blocked by the cap interference inner circumferential portion 126, whereby the disc portion 131 cannot pass through the through-hole 125. In this state, when the user rotates the hollow closure cap 130 by a certain angle about the axial line AX, the plurality of holder interference protrusions 132 overlaps the cap interference inner circumferential portion 126, whereby the hollow closure cap 130 is fixedly coupled to the second filter holder 123 such that that the hollow closure cap 130 is not separated from the through-hole 125. When the user pulls down or lifts up the handle 134 while holding the handle 134 in the state in which the hollow closure cap 130 is fixedly coupled to the second filter holder 123, as described above, the filter module 110 is moved downward or upward along with the hollow closure cap 130.

The solid air freshener pocket 135 is a cylindrical portion having a space, in which a volatile solid air freshener (not shown) is received, formed therein, and an approximately disc-shaped pocket coupling cap 138 removably coupled to an upper end of the solid air freshener pocket 135. A plurality of pocket sidewall through-holes 136 configured to allow air to flow therethrough is formed in a sidewall of the solid air freshener pocket 135. In order to receive a consumable solid air freshener in the pocket coupling cap 138, the user may separate the pocket coupling cap 138 from the solid air freshener pocket 135, may insert the solid air freshener into the solid air freshener pocket 135, and may couple the pocket coupling cap 138 to the upper end of the solid air freshener pocket 135 again.

The ultraviolet radiator 186 is fixedly attached to the ultraviolet radiator support 160 in the case 150, is disposed above the photocatalytic filter 140 so as to face the photocatalytic filter 140, and is disposed under the fan 183.

The ultraviolet radiator 186 includes a circular light emitting diode (LED) PCB 187, a plurality of ultraviolet A LEDs 190 and a plurality of ultraviolet C LEDs 191 electrically mounted on a lower surface of the LED PCB 187. The plurality of ultraviolet A LEDs 190 and the plurality of ultraviolet C LEDs 191 are installed at positions to radiate ultraviolet A having a wavelength of 315 to 400 nm and ultraviolet C having a wavelength of 100 to 280 nm while facing the photocatalytic filter 140 having the titanium oxide layer formed on the surface thereof.

When ultraviolet A radiated by the plurality of ultraviolet A LEDs 190 is incident on the titanium oxide layer, OH radicals are generated. The OH radicals cling to viruses, odor molecules, and harmful bacteria in the air and decompose the viruses, the odor molecules, and the harmful bacteria to remove the viruses, the odor molecules, and the harmful bacteria. Consequently, the air purifier 101 has excellent performance in removing viruses, odor molecules, and harmful bacteria in addition to removing general pollutants contained in indoor air. The ultraviolet C radiated by the plurality of ultraviolet C LEDs 191 sterilizes germs contained in the air that has passed through the photocatalytic filter 140.

Air introduced into the hollow 112 of the dust collection filter 111 includes a volatile aromatic component of the solid air freshener (not shown) received in the solid air freshener pocket 135, whereby a room in which the air freshener 101 is operated may be filled with a scent that enhances the mood of the user. The LED PCB 187 is provided with a through-hole 188 configured to allow air to flow therethrough upward and downward. In the ultraviolet radiator provided in the present invention, the shape and number of the through-hole 188 are not limited to the embodiment shown in FIGS. 2 and 3. Meanwhile, the ultraviolet radiator support 160 is also provided with a through-hole 162 aligned with the through-hole of the LED PCB 187 up and down. Consequently, air that has moved upward from the hollow 112 of the dust collection filter 111 and has passed through the plurality of pores 142 of the photocatalytic filter 140 moves upward through the through-hole 188 of the LED PCB 187 and the through-hole 162 of the ultraviolet radiator support 160.

The air purifier 101 further includes a permanent magnet 164 disposed above the LED PCB 187 in the case, wherein attractive force is applied between the permanent magnet 129 provided at the filter module 110 and the permanent magnet disposed above the LED PCB. Specifically, six magnet installation recesses (not shown) may be formed in a bottom surface of the ultraviolet radiator support 160, and six permanent magnets 164 may be fixedly installed in the magnet installation recesses, respectively. The six permanent magnets 164 are disposed so as to be spaced apart from each other by the same angle based on the axial line AX. A lower surface of each of the permanent magnets 164 may be in contact with an upper surface of the LED PCB 187 or may be adjacent to the upper surface of the LED PCB 187 so as to be nearly in contact therewith.

The horizontal distance from the axial line AX of the filter module 110 to each of the permanent magnets 164 disposed above the ultraviolet radiator 186 and the horizontal distance from the axial line AX of the filter module 110 to each of the permanent magnets 129 belonging to the filter module 110 are equal to each other. The six permanent magnets 164 disposed above the ultraviolet radiator 186 and the six permanent magnets 129 provided at the filter module 110 are disposed such that attractive force is applied therebetween. For example, if a lower surface of each of the six permanent magnets 164 disposed above the ultraviolet radiator 186 is N-polar, an upper surface of each of the six permanent magnets 129 of the filter module 110 is S-polar. Conversely, if the lower surface of the six permanent magnets 164 disposed above the ultraviolet radiator 186 is S-polar, the upper surface of the six permanent magnets 129 of the filter module 110 is N-polar.

The first filter holder 113 is removably attached to the LED PCB 186 by attractive force between the six permanent magnets 164 disposed above the ultraviolet radiator 186 and the six permanent magnets 129 of the filter module 110. In other words, when the user inserts the filter module 110 into the case 150 and raises the filter module 110 from below to above the ultraviolet radiator 186, the six permanent magnets 164 disposed above the ultraviolet radiator 186 and the six permanent magnets 129 of the filter module 110 are attracted to each other one-to-one, whereby the filter module 110 is removably attached to the LED PCB 187 of the ultraviolet radiator 186 so as to be supported thereby.

The user may rotate the filter module 110 by 60° based on the axial line AX to change the attitude of the filter module 110 attached to the LED PCB 187. When the first filter holder 113 of the filter module 110 is attached to the LED PCB 187, upper ends of the annular sidewalls 114w and 117w of the first and second members 114 and 117 are in contact with the LED PCB 187 while avoiding the plurality of ultraviolet A LEDs 190 and the plurality of ultraviolet C LEDs 191, and the photocatalytic filter 140 is also spaced apart from the LED PCB 187, whereby the plurality of ultraviolet A LEDs 190, the plurality of ultraviolet C LEDs 191, and the photocatalytic filter 140 are not damaged. Meanwhile, when the user holds the handle 134 of the hollow closure cap 130 of the filter module 110 attached to the LED PCB 187 and pulls the handle downward with force greater than attractive force between the permanent magnets 129 and 164, the filter module 110 may be separated from the LED PCB 187 and may be removed outward from the interior of the case 150.

The air purifier 101 further includes a pre-filter 165 disposed below the filter module 110, specifically below the second filter holder 123, in the case 150. Foreign matter having a relatively large particle size contained in air outside the air purifier 101, such as dust and yellow dust, is filtered out by the pre-filter 165 prior to being filtered out through the dust collection filter 111. Accordingly, the pre-filter 165 may be referred to as a primary dust collection filter, and the dust collection filter 111 provided in the filter module 110 may be referred to as a secondary dust collection filter.

The primary dust collection filter 165 includes a primary dust collection filter sheet 168 made of fabric, a primary dust collection filter bracket 166 configured to support the primary dust collection filter sheet 168 in an unfolded state, and a plurality of spacers 167 projecting upward from an outer circumferential portion of the primary dust collection filter bracket 166. The primary dust collection filter sheet 168 is spaced apart from the second filter holder 123 of the filter module 110 by a certain distance due to the plurality of spacers 167.

The primary dust collection filter 165 may be supported by the inner wall 157 in the case 150, or may be disposed above the inner wall 157 in a state of being spaced apart from the inner wall 157. The inner wall 157 is provided with a through-hole 158 aligned with the primary dust collection filter 165 up and down.

When the fan 183 of the air purifier 101 is operated, air introduced into the case 150 through the air inlets 155 and 156, as indicated by solid arrows shown in FIG. 1, passes through the through-hole 158 of the interior wall 157 and the primary dust collection filter 165. The air having passed through the primary dust collection filter 165 is blocked by the hollow closure cap 130, is redirected in a radial direction to rise between the inner circumferential surface of the sidewall 152 of the case 150 and the outer circumference of the secondary dust collection filter 111, passes through the secondary dust collection filter 111, and moves toward the interior of the hollow 112. In addition, the air introduced into the hollow 112 moves upward, is discharged from the secondary dust collection filter 111, successively passes through the photocatalytic filter 140, the ultraviolet radiator 186, and the through-hole 162 of the ultraviolet radiator support 160, is moved upward by the fan propeller 185 of the fan 183 in a pressurized state, and is discharged out of the case 150 through the air outlet 153.

The air purifier 101 may further include a battery configured to supply electrical energy to the fan 183 and LED PCB 187 disposed in the case 150, and a contaminant sensor configured to detect contaminants contained in the air introduced from the outside.

The filter module 110 described above includes a dust collection filter 111 and a photocatalytic filter 140 configured to allow air that has passed through the dust collection filter 111 to pass therethrough, wherein the photocatalytic filter 140 includes a titanium oxide layer at which OH radicals are generated when ultraviolet A is incident thereon. Consequently, the filter module 110 and an air purifier 101 including the same have excellent performance in removing foreign matter, such as fine dust, yellow dust, and dust, from the air and excellent performance in removing viruses, odor molecules, and harmful bacteria.

The present invention has been described with reference to the embodiment shown in the drawings, which is exemplary only, and a person having ordinary skill in the art to which the present invention pertains will recognize that various modifications and equivalents are possible therefrom. Accordingly, the true scope of the present invention should be determined only by the appended claims.

1. A filter module comprising:
a dust collection filter, which is a tubular filter having a hollow formed therein, the dust collection filter being configured to filter out contaminants from air flowing from outside an outer circumference thereof toward an interior of the hollow;
a photocatalytic filter configured to allow air introduced into the hollow and discharged out of the dust collection filter through an opening above the hollow to pass therethrough, the photocatalytic filter comprising a pore plate having a plurality of pores formed therein and a titanium oxide layer formed by laminating titanium oxide on a surface of the pore plate through which the air is discharged;
a first filter holder configured to fixedly support the photocatalytic filter and an upper end of the dust collection filter so as not to obstruct air flowing from the interior of the hollow to the photocatalytic filter; and
a second filter holder configured to fixedly support a lower end of the dust collection filter.

2. The filter module according to claim 1, wherein the pore plate is made of porous nickel foam.

3. The filter module according to claim 1, further comprising:
a hollow closure cap removably coupled to the second filter holder, the hollow closure cap being configured to close an opening under the hollow, wherein
the hollow closure cap comprises a solid air freshener pocket configured to allow a solid air freshener to be received therein.

4. The filter module according to claim 3, wherein
the hollow closure cap further comprises a downwardly projecting handle, and
the filter module is pulled down along with the handle when the handle is pulled down in a state in which the hollow closure cap is fixedly coupled to the second filter holder.

5. An air purifier comprising:
a case having an air inlet formed at a side and an air outlet formed at a top;
the filter module according to claim 1, the filter module being disposed in the case, the filter module being configured to purify air introduced into the case through the air inlet;
a fan disposed in the case, the fan being configured to move air such that the air is introduced into the case through the air inlet, passes through the filter module, rises, and is discharged out of the case through the air outlet; and
an ultraviolet radiator disposed above the photocatalytic filter so as to face the photocatalytic filter in the case, the ultraviolet radiator comprising an ultraviolet A light emitting diode (LED) configured to radiate ultraviolet A to the titanium oxide layer such that OH radicals are generated at the titanium oxide layer and an LED printed circuit board (PCB) on which the ultraviolet A LED is electrically mounted.

6. The air purifier according to claim 5, wherein the LED PCB is provided with a through-hole configured to allow air to flow therethrough.

7. The air purifier according to claim 5, wherein the ultraviolet radiator further comprises an ultraviolet C LED configured to radiate ultraviolet C used to sterilize air that has passed through the photocatalytic filter, the ultraviolet C LED being electrically mounted on the LED PCB.

8. The air purifier according to claim 5, wherein
the filter module further comprises a permanent magnet fixedly installed at the first filter holder,
the air purifier comprises a permanent magnet disposed above the LED PCB in the case, attractive force being applied between the permanent magnet of the filter module and the permanent magnet disposed above the LED PCB, and
the first filter holder is removably attached to the LED PCB by the attractive force between the permanent magnet of the filter module and the permanent magnet disposed above the LED PCB.
